(19) United States
(12) Patent Application Publication
(10) Pub. No.: US 2021/0343410 A1
(43) Pub. Date: Nov. 4, 2021

(54) Method to the automatic International Classification of Diseases (ICD) coding for clinical records

(71) Applicant: Petuum Inc., Pittsburgh, PA (US)

(72) Inventors: Shanghang Zhang, Mountain View, CA (US); Najmeh Sadoughi, Pittsburgh, PA (US); Pengtao Xie, Pittsburgh, PA (US); Eric Xing, Pittsburgh, PA (US)

(73) Assignee: Petuum Inc., Pittsburgh, PA (US)

(21) Appl. No.: 16/865,335

(22) Filed: May 2, 2020

(51) Int. Cl. G16H 50/20; G06F 40/30; G16H 50/70; G16H 10/60; G16H 70/60; G16H 40/20; G06N 3/04; G06N 3/08; G06F 16/22

(52) U.S. Cl. CPC G16H 50/20; G06F 40/30; G16H 50/70; G16H 10/60; G06F 16/2246; G16H 40/20; G06N 3/0454; G06N 3/0445; G06N 3/088; G16H 70/60

(57) ABSTRACT

The present invention is a system and a method to classify clinical records into International Classification of Diseases (ICD) codes. The system includes a processor, and a memory communicatively coupled to the processor. The memory includes a generator (G), a feature extractor, a discriminator (D), a label encoder, and a keywords reconstructor. The generator (G) generates synthesized features corresponding to ICD code descriptions. The feature extractor extracts real latent features from clinical documents and generates real features by training a GANs. The generator (G) generates synthesized features after the GANs are trained and calibrate a binary code classifier with the real latent features generated by the feature extractor for a low-shot ICD code l. The feature extractor generates code-specific latent features conditioned on a textual description of each ICD code description by using a WGAN-GP. The discriminator (D) distinguishes between the synthesized features and the real features and determines whether the features are the real features or synthetic features. The label encoder encodes a sequence of keywords in the ICD code description into a sequence of hidden states.

### TECHNICAL FIELD

The present invention relates to data analysis and processing, in particular to system and method to classify clinical records into the International Classification of Diseases (ICD) codes.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in-and-of-themselves may also be inventions.

Typically, patient interactions with health care providers such as hospitals, clinics, or doctors are being digitized at a rapidly accelerated pace. The digital records of these patient interactions include data regarding early presentations of symptoms, sets of diagnostic tests administered and their results, passive monitoring results, series of interventions, and detailed reports of health progression by health practitioners. The diagnosis and procedures are classified for the unification of the digital records. The International Classification of Diseases (ICD) is a list of classification codes for the diagnosis. In healthcare facilities, clinical records are classified into a set of ICD codes that categorize diagnosis and procedures. ICD codes are used for a wide range of purposes including billing, reimbursement, and retrieving of diagnostic information. Automatic ICD coding is in great demand as manual coding can be labor-intensive and error-prone.

This specification recognizes that there is a need for a system and method to automatically and accurately classify the patients' clinical notes into ICD codes. Automatic ICD coding is a multi-label text classification task with an extremely long-tailed class label distribution, making it difficult to perform fine-grained classification on both frequent and infrequent ICD codes at the same time. The majority of ICD codes only have a few or no labelled data due to the rareness of the disease. In the existing medical dataset such as MIMIC III, among 17,000 unique ICD-9 codes, more than 50% of them never occur in the training data. It is extremely challenging to perform fine-grained multi-label classification on both codes with sufficient labelled data (frequent codes, e.g., with approximately greater than 20 labelled samples), and insufficient labelled data (low-shot codes, e.g., approximately 0 to 20 labelled samples), at the same time. Automatic ICD coding for both frequent codes and low-shot codes fit into the generalized low-shot learning (GLSL) paradigm, where test examples are from both frequent and low-shot classes and there is a need to classify them into the joint labelling space of both types of classes. Nevertheless, current GLSL works focus on visual tasks. There are few existing systems and methods on GLSL for multi-label text classification. Further, the existing automatic ICD coding models assign frequent ICD codes while performing quite poorly on low-shot codes.

To resolve the above discrepancy, there is a need to improve the predictive power on both frequent and low-shot codes by fine-tuning the models with synthetic latent features. The official ICD guidelines provide each code with a short text description and a hierarchical tree structure on all the ICD codes (ICD-9 Guidelines). Further, there is a need for a system and a method to exploit this domain knowledge about ICD codes to generate semantically meaningful features. Several approaches have explored automatic assigning of ICD codes on clinical text data. The existing system and method extract per-code textual features with attention mechanisms for ICD code assignments. Additionally, the existing system and method explored character-based long short-term memory (LSTM) with attention. Also, the existing system and method apply tree LSTM with ICD hierarchy information for ICD coding. These systems and methods do not assign rare codes in their final prediction, making it impractical to deploy in real applications.

Automatic ICD coding is a multi-label text classification task with noisy clinical document inputs and extremely long-tailed label distribution. ICD coding for both frequent and low-shot codes fits into the generalized low-shot learning (GLSL) paradigm. Furthermore, the existing system and method explore low-shot text classification by learning the relationship between text and weakly labelled tags on a large corpus. However, these approaches cannot be directly applied to ICD coding as the input is labelled with a set of codes that can include both frequent and low-shot codes. Note that, it is often not possible to determine ahead of time (e.g., prior to training or learning) if the data is from a frequent or a low-shot class for ICD coding.

Thus, in view of the above, there is a long-felt need in the industry to address the aforementioned deficiencies and inadequacies.

Further limitations and disadvantages of conventional and traditional approaches will become apparent to one of ordinary skill in the art through comparison of described systems with some aspects of the present disclosure, as set forth in the remainder of the present application and with reference to the drawings.

### SUMMARY

A system and method for classifying clinical records into the International Classification of Diseases (ICD) codes are provided substantially, as shown in and/or described in connection with at least one of the figures.

An aspect of the present invention relates to a system to classify a plurality of clinical records into the International Classification of Diseases (ICD) codes. The system includes one or more processor(s), and a memory communicatively coupled to the processor(s). The memory stores instructions that can be executed by the processor(s), and when the stored instructions are executed by the processor(s) they cause the processor(s) to perform one or more steps of classifying a plurality of clinical records into ICD codes described herein. The memory includes a generator (G), a feature extractor, a discriminator (D), a label encoder, and a keywords reconstructor. The generator (G) generates one or more synthetic features corresponding to one or more ICD code descriptions. In an aspect, the synthetic features are formed by multiplying or crossing two or more ICD code descriptions. The multiplied combinations of ICD code descriptions can provide predictive abilities beyond what those ICD code descriptions can provide individually. The feature extractor extracts one or more real latent features from a plurality of clinical documents and generates one or more real features by training a plurality of generative adversarial networks (GANs). According to an aspect herein, the real latent features are a representation of compressed data of the clinical documents. In an aspect, the generator (G) generates synthesized features after the GANs are trained and calibrates or fine-tunes a binary code classifier with the real latent features generated by the feature extractor for a low-shot ICD code l. According to an aspect herein, the binary code classifier matches each of the real latent features data with a label and classifies the real latent features into either zero or one. In an aspect, the binary code classifier is encoded by a graph gated recurrent neural networks (GRNN) to classify the real latent features into either zero or one.

The GANs improve the low-shot ICD code l by generating a plurality of pseudo data examples in a latent feature space of the clinical documents for the low-shot ICD codes 1. The generator (G) generates one or more code-specific latent features conditioned on a textual description of each ICD code description by using a Wasserstein GAN with a gradient penalty (WGAN-GP). The Wasserstein GAN with gradient penalty (WGAN-GP) generates a latent feature vector (f). The discriminator (D) distinguishes between the synthesized features generated by the generator (G) and the real features generated by the feature extractor and determines whether the features are the real features generated by feature extractor or the synthetic features generated by the generator (G). The label encoder encodes a sequence of a plurality of keywords or M words in the ICD code description into a sequence of one or more hidden state sequences by using a long short-term memory (LSTM). In an aspect, the label encoder obtains a fixed-sized encoding vector (el) by performing a dimension-wise max-pooling over the hidden state sequences. In an aspect, the label encoder obtains an eventual embedding (cl=el∥gl) of the code l by concatenating the fixed-sized encoding vector (el) with an ICD tree hierarchy (gl) which is the embedding of the code l produced by a graph encoding network. In an aspect, the eventual embedding (cl) includes a latent semantics of the description (in el) and the ICD tree hierarchy (in gl). The keywords reconstructor reconstructs the keywords extracted from the clinical documents associated with a code l to ensure the latent feature vector (f̅) captures a semantic meaning of a code l. For the code l, a long short-term memory (LSTM is used to encode the sequence of M words in the description into a sequence of hidden states [e1, e2, . . . , eM]. Then a dimension-wise max-pooling is performed over the hidden state sequence to get a fixed-sized encoding vector el to obtain the eventual embedding cₗ=el∥gl of code l by concatenating el with gl which is the embedding of l produced by the graph encoding network. Cl contains both the latent semantics of the description (in el) as well as the ICD hierarchy information (in gl).

The distinction between codes with sufficient labelled data and codes insufficient labelled data may depend on the particular use case circumstances. In some embodiments, codes with sufficient labelled data are codes with one or more labelled data and codes with insufficient labelled data are codes with 0 labelled data (also called zero-shot data). In some embodiments, codes with sufficient labelled data are codes with greater than 20 labelled data and codes with insufficient labelled data are codes with approximately 0 to 20 labelled data. In some embodiments, codes with sufficient labelled data are codes with greater than 30 labelled data and codes with insufficient labelled data are codes with approximately 0 to 30 labelled data. In some embodiments, codes with sufficient labelled data are codes with greater than 40 labelled data and codes with insufficient labelled data are codes with approximately 0 to 40 labelled data. In some embodiments, codes with sufficient labelled data are codes with greater than 50 labelled data and codes with insufficient labelled data are codes with approximately 0 to 50 labelled data.

Another aspect of the present invention relates to a method for classifying a plurality of clinical records into the International Classification of Diseases (ICD) codes. The method includes the step of generating one or more synthetic features corresponding to one or more ICD code descriptions through a generator (G). The method includes the step of extracting one or more real latent features from a plurality of clinical documents and generating one or more real features by training a plurality of generative adversarial networks (GANs) through a feature extractor. The generator (G) synthesizes the real features after the GANs are trained and calibrates or fine-tunes a binary code classifier with the real latent features generated by the feature extractor for a low-shot ICD code l. The GANs improve the low-shot ICD code l by generating a plurality of pseudo data examples in a latent feature space of the clinical documents for the low-shot ICD codes l. The feature extractor generates one or more code-specific latent features conditioned on a textual description of each ICD code description by using a Wasserstein GAN with a gradient penalty (WGAN-GP). The Wasserstein GAN with gradient penalty (WGAN-GP) generates a latent feature vector (f). The method includes the step of distinguishing between the synthetic features generated by the generator (G) and the real features generated by the feature extractor and determining whether the features are a real feature or a synthetic feature through a discriminator (D). The method includes the step of encoding a sequence of a plurality of keywords (M words) in the ICD code description into a sequence of one or more hidden state sequences by using a long short-term memory (LSTM) through a label encoder. The method includes the step of reconstructing the keywords extracted from the clinical documents associated with a code l for ensuring the latent feature vector (f̅) captures a semantic meaning of a code l through a keywords reconstructor. The method includes the step of obtaining a fixed-sized encoding vector (el) by performing a dimension-wise max-pooling over the hidden state sequences through the label encoder. The method includes the step of obtaining an eventual embedding (cl=el∥gl) of the code l by concatenating the fixed-sized encoding vector (el) with an ICD tree hierarchy (gl) which is the embedding of the code l produced by a graph encoding network through the label encoder.

In an aspect, the eventual embedding (cl) includes a latent semantics of the description (in el) and the ICD tree hierarchy (in gl). According to an aspect herein, the latent semantic provides the underlying meaning of the keywords extracted from the clinical documents.

In an aspect, the binary code classifier is encoded by a graph gated recurrent neural networks (GRNN).

Accordingly, one advantage of the present invention is that it provides an adversarial generative model AGM-HT for automatic ICD coding.

Accordingly, one advantage of the present invention is that the AGM-HT generates latent features conditioned on the code descriptions and fine-tunes the low-shot ICD code assignment classifiers.

Accordingly, one advantage of the present invention is that the AGM-HT exploits the hierarchical structure of ICD codes to generate semantically meaningful features for zero-shot codes without any labelled data. To further facilitate the feature synthesis of low-shot ICD codes, the low-shot ICD codes are encouraged to generate similar features with their nearest sibling code according to the hierarchical structure of the ICD codes. In order to train the generator (G) and discriminator for zero-shot codes, the ICD hierarchy is utilized and used f_{sib}, the latent feature extracted from real data of the nearest sibling I_{sib} of a zero-shot code I, for training the discriminator. The WGAN distance between f_{sib} and the generated feature is minimized to make the generated feature f⁻ to be close to the real latent features of the siblings of I and thus f⁻ can better preserve the ICD hierarchy.

Accordingly, one advantage of the present invention is that the AGM-HT includes a pseudo cycle generation architecture to guarantee the semantic consistency between the synthetic and real features by reconstructing the relevant keywords in input documents.

Accordingly, one advantage of the present invention is that it improves the F1 score from nearly 0 to 20.91% for the low-shot codes and AUC score by 3% (absolute improvement) on a MIMIC-III dataset from the previous state of the arts.

Accordingly, one advantage of the present invention is that the AGM-HT improves the performance of few-shot codes with a handful of labelled data.

Other features of embodiments of the present invention will be apparent from accompanying drawings and from the detailed description that follows.

Yet other objects and advantages of the present invention will become readily apparent to those skilled in the art following the detailed description, wherein the preferred embodiments of the invention are shown and described, simply by way of illustration of the best mode contemplated herein for carrying out the invention. As we realized, the invention is capable of other and different embodiments, and its several details are capable of modifications in various obvious respects, all without departing from the invention. Accordingly, the drawings and description thereof are to be regarded as illustrative in nature, and not as restrictive.

### BRIEF DESCRIPTION OF THE DRAWINGS

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label irrespective of the second reference label.

FIG. 1 illustrates a network implementation of the present system to classify a plurality of clinical records into the International Classification of Diseases (ICD) codes, in accordance with one embodiment of the present invention.

FIG. 2 illustrates a block diagram of the various components of the memory of the present system, in accordance with one embodiment of the present invention.

FIG. 3 illustrates a block diagram of the present system to classify a plurality of clinical records into the International Classification of Diseases (ICD) codes, in accordance with one embodiment of the present invention.

FIG. 4 illustrates a flowchart of the method for classifying a plurality of clinical records into the International Classification of Diseases (ICD) codes, in accordance with an alternative embodiment of the present invention.

### DETAILED DESCRIPTION

The present invention is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond certain implementation choices in the following embodiments.

Systems and methods are disclosed for classifying a plurality of clinical records into the International Classification of Diseases (ICD) codes. Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, firmware, and/or by human operators.

Embodiments of the present invention may be provided as a computer program product, which may include a machine-readable storage medium tangibly embodying thereon instructions, which may be used to program a computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

Various methods described herein may be practiced by combining one or more machine-readable storage media containing the code according to the present invention with appropriate standard computer hardware to execute the code contained therein. An apparatus for practicing various embodiments of the present invention may involve one or more computers (or one or more processors within a single computer) and storage systems containing or having network access to computer program(s) coded in accordance with various methods described herein, and the method steps of the invention could be accomplished by modules, routines, subroutines, or subparts of a computer program product.

The present invention discloses a system and method, whereby an adversarial generative model conditioned on code descriptions with a hierarchical tree structure (AGM-HT) to generate synthetic features. The present system and method improve the predictive power on both frequent and low-shot codes by fine-tuning the models with synthetic latent features. In various embodiments, low-shot codes may include 0 labelled data, or 0 to 5 labelled data, 0 to 10 labelled data, 0 to 15 labelled data, 0 to 20 labelled data, 0 to 25 labelled data, 0 to 30 labelled data, 0 to 40 labelled data, or 0 to 50 labelled data.

Although the present invention has been described with the purpose of the automatic International Classification of Diseases (ICD) coding for clinical records, it should be appreciated that the same has been done merely to illustrate the invention in an exemplary manner and to highlight any other purpose or function for which explained structures or configurations could be used and is covered within the scope of the present invention.

The term “machine-readable storage medium” or “computer-readable storage medium” includes, but is not limited to, portable or non-portable storage devices, optical storage devices, and various other mediums capable of storing, containing, or carrying instruction(s) and/or data. A machine-readable medium may include a non-transitory medium in which data can be stored, and that does not include carrier waves and/or transitory electronic signals propagating wirelessly or over wired connections. Examples of a non-transitory medium may include but are not limited to, a magnetic disk or tape, optical storage media such as compact disk (CD) or versatile digital disk (DVD), flash memory, memory or memory devices.

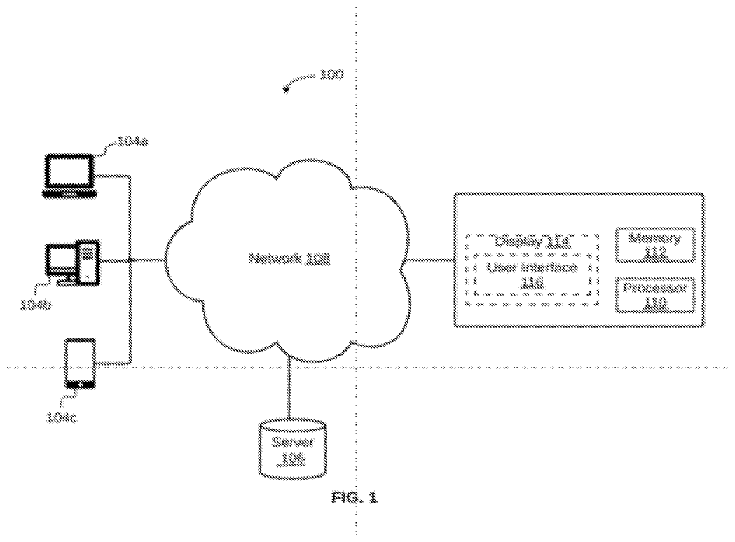

FIG. 1 illustrates a network implementation of the present system 100 to classify a plurality of clinical records into the International Classification of Diseases (ICD) codes, in accordance with one embodiment of the present invention. The system 100 includes a processor 110, and a memory 112 communicatively coupled to the processor 110. The memory 112 stores instructions executed by the processor 110. Although the present subject matter is explained considering that the present system 100 is implemented on a server 106, it may be understood that the present system 100 may also be implemented in a variety of computing devices 104, such as a laptop computer 104a, a desktop computer 104b, a smartphone 104c, a notebook, a workstation, a mainframe computer, server, a network server, and the like. It will be understood that the present system 100 may be accessed by multiple users through the computing devices collectively referred to as computing device 104 hereinafter, or applications residing on the computer devices 104. Examples of the computing devices 104 may include but are not limited to, a portable computer, a personal digital assistant, a handheld device, and a workstation. The computing devices 104 are communicatively coupled to a network 108 and utilizes the various operating system to perform the functions of the present system 100 such as Android, IOS, Windows, etc.

In one implementation, the network 106 may be a wireless network, a wired network, or a combination thereof. The network 106 can be implemented as one of the different types of networks, such as an intranet, local area network (LAN), wide area network (WAN), the internet, and the like. The network 106 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further, the network 106 may include a variety of network devices, including routers, bridges, servers, computing devices, storage devices, and the like. When a user of laptop 104a, for example, wants to visualize classified a plurality of clinical records, laptop 104a communicates the same with the server 106, via network 108. The server 106 then presents the classified clinical records as per the user's request. The server 106 is a computer or computer program that manages access to a centralized resource or service in the network 108.

The processor 110 is communicatively coupled to the memory 112, which may be a non-volatile memory or a volatile memory. Examples of non-volatile memory may include, but are not limited to flash memory, a Read Only Memory (ROM), a Programmable ROM (PROM), Erasable PROM (EPROM), and Electrically EPROM (EEPROM) memory. Examples of volatile memory may include but are not limited Dynamic Random Access Memory (DRAM), and Static Random-Access memory (SRAM).

Processor 110 may include at least one data processor for executing program components for executing user- or system-generated requests. A user may include a person, a person using a device such as those included in this invention, or such a device itself. Processor 110 may include specialized processing units such as integrated system (bus) controllers, memory management control units, floating point units, graphics processing units, digital signal processing units, etc.

Processor 110 may include a microprocessor, such as AMD® ATHLON® microprocessor, DURON® microprocessor OR OPTERON® microprocessor, ARM's application, embedded or secure processors, IBM® POWERPC®, INTEL'S CORE® processor, ITANIUM® processor, XEON® processor, CELERON® processor or other line of processors, etc. Processor 110 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor 110 may be disposed of in communication with one or more input/output (I/O) devices via an I/O interface. I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

The present system 100 further includes a display 114 having a User Interface (UI) 116 that may be used by the user or an administrator to initiate a request to view the classified clinical records. Display 114 further be used to display the classified plurality of clinical records.

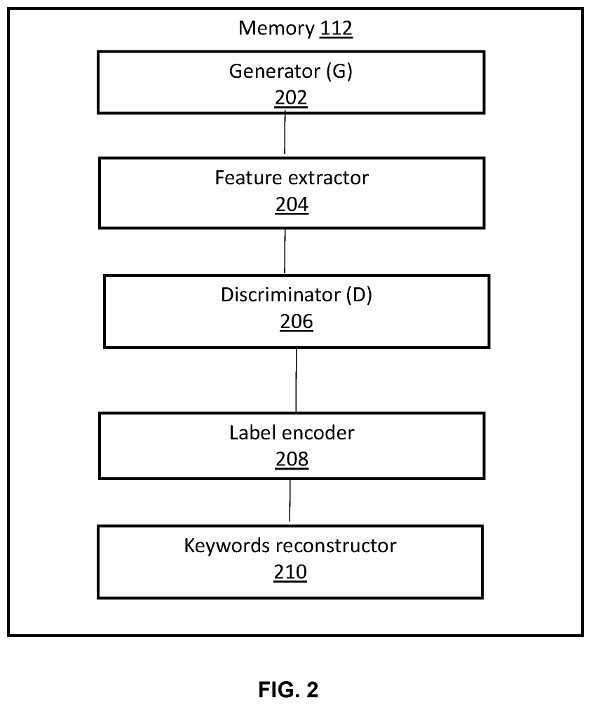

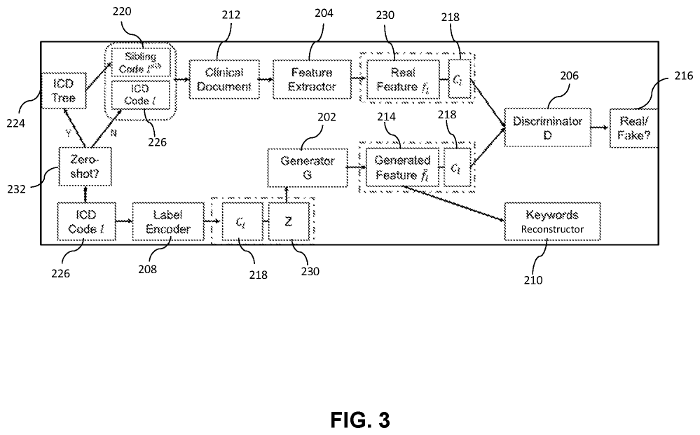

FIG. 2 illustrates a block diagram of the various components of the memory 112 of the present system, in accordance with one embodiment of the present invention. The memory 112 includes a generator (G) 202, a feature extractor 204, a discriminator (D) 206, a label encoder 208, and a keywords reconstructor 210. FIG. 2 is explained in conjunction with FIG. 3. The generator (G) 202 generates one or more features (f̅ₗ) corresponding to one or more ICD code l descriptions 226. The feature extractor 204 extracts one or more real latent features (fₗ) 230 from a plurality of clinical documents 212 and generates one or more real features by training a plurality of generative adversarial networks (GANs). In an embodiment, the generator (G) 202 synthesizes the real features after the GANs are trained and calibrates or fine-tunes a binary code classifier with the real latent features (fₗ) 230 generated by the feature extractor 204 for a low-shot ICD code l. In an embodiment, the binary code classifier is encoded by a graph gated recurrent neural networks (GRNN).

The GANs improve the low-shot ICD code l 232 by generating a plurality of pseudo data examples in a latent feature space of the clinical documents 212 for the low-shot ICD codes l. In an embodiment, the GANs generate features for both zero and few shot codes. So, “Y” after 232 means that that the sibling codes can also be used for training the GANs. According to an embodiment herein, the low-shot ICD code l can be replaced by zero-shot ICD code l. The feature extractor 204 generates one or more code-specific latent features conditioned on a textual description of each ICD code description by using a Wasserstein GAN with a gradient penalty (WGAN-GP). The Wasserstein GAN with gradient penalty (WGAN-GP) generates a latent feature vector (f̅). The discriminator (D) 206 distinguishes between the features generated by the generator (G) 202 and the real features generated by the feature extractor 204 and determines whether the features are a real feature or a fake feature 216. The label encoder 208 encodes a sequence of a plurality of keywords or M words in the ICD code description into a sequence of one or more hidden state sequences by using a long short-term memory (LSTM). In an embodiment, the label encoder 208 obtains a fixed-sized encoding vector (el) by performing a dimension-wise max-pooling over the hidden state sequences. In an embodiment, the label encoder 208 obtains an eventual embedding 218 (cl=el∥gl) of the code l by concatenating the fixed-sized encoding vector (el) with an ICD tree hierarchy (gl) which is the embedding of the code l produced by a graph encoding network. In an embodiment, the eventual embedding (cl) 218 includes a latent semantics of the description (in el) and the ICD tree hierarchy (in gl) 224. The keywords reconstructor 210 reconstructs the keywords extracted from the clinical documents 212 associated with a code l to ensure the latent feature vector (f̅) captures a semantic meaning of a code l.

FIG. 3 illustrates a block diagram 300 of the present system to classify a plurality of clinical records into the International Classification of Diseases (ICD) codes, in accordance with one embodiment of the present invention. The present system presents an adversarial generative model conditioned on code descriptions with a hierarchical tree structure for automatic ICD coding (AGM-HT). To solve the automatic ICD coding problem, the present system provides AGM-HT, an Adversarial Generative Model conditioned on code descriptions with Hierarchical Tree structure to generate synthetic features. The AGM-HT includes a generator 202 to synthesize code-specific latent features based on the ICD code descriptions, and a discriminator 206 to decide how realistic the generated features are. To guarantee the semantic consistency between the generated and real features, AGM-HT reconstructs the keywords in the input documents that are relevant to the conditioned codes. To further facilitate the feature synthesis of low-shot codes, the hierarchical structure of the ICD codes utilized to encourage the low-shot codes to generate similar features with their nearest sibling code l^{sub} 220. The ICD coding models are fine-tuned on the generated features to achieve a more accurate prediction for low-shot codes. According to an embodiment herein, the ICD coding model is a classifier model.

The classifier model is composed of a feature extractor which is shared between all the labels, and an attention layer followed by a graph encoded binary layer for classification. After training the GAN for the low-shot classes, the present system utilizes the generated features of low-shot codes and their corresponding labels to train the graph encoded binary layer of the classifier again.

The task of automatic ICD coding is to assign ICD codes l 226 to patient's clinical documents. During the experiment, a problem has been formulated as a multi-label text classification problem. Let L be the set of all ICD codes and l=|L|, given an input text, the goal is to predict yl∈{0, 1} for all l∈L. Each ICD code l has a short text description. For example, the description for ICD-9 code 403.11 is “Hypertensive chronic kidney disease, benign, with chronic kidney disease stage V or end-stage renal disease.” There is also a known hierarchical tree structure on all the ICD codes: for a node representing an ICD code, the children of this node represent the subtypes of this ICD code. Among all the ICD codes, some codes have a lot of samples in the training set, while some codes have only a few or no samples in the training set. Automatic ICD coding has to classify both frequent codes and low-shot codes at the same time, which is a generalized low-shot ICD coding problem. This invention effectively solves the generalized low-shot ICD coding problem by accurately assigning code l given that l is never assigned to any training text (assigned only to a few training texts), without sacrificing the performance on codes with training data.

During the experiment, a pre-trained model is assumed as a feature extractor that performs ICD coding by extracting label-wise feature fₗ and predicting yₗ by σ(gₗ^{τ}·fₗ), where σ is the sigmoid function and gₗ is the binary classifier for code l. For the low-shot codes, gₗ is never trained (trained only a few times) on fₗ with yₗ=1 and thus at inference time, the pre-trained feature extractor hardly ever assigns low-shot codes. The present system and method use a generative adversarial network (GAN) to generate \tilde{f}ₗ with yₗ=1 by conditioning on code l. FIG. 3 shows an overview of the generation framework. The generator G 202 tries to generate the fake feature given an ICD code description. The discriminator D 206 tries to distinguish between the generated feature and the real latent feature from the feature extractor model. After the GAN is trained, the generator G 202 synthesizes the feature and fine-tunes the binary classifier with the generated feature for a given low-shot code l. Since the binary code classifiers are independently fine-tuned for low-shot codes, the performance on the frequent codes is not affected, achieving the goal of generalized low-shot ICD coding.

In an embodiment, the pre-trained feature extractor model is low-shot attentive graph recurrent neural networks (LA-GRNN) modified from low-shot attentive graph convolution neural networks (LAGCNN), which is the only previous work that is tailored towards solving low-shot ICD coding. The present system and method improve the original implementation by replacing the GCNN with graph gated recurrent neural networks (GRNN) and adopting the label-distribution-aware margin loss for training. At a high-level, given an input x, LAGRNN extracts label-wise feature fₗ and performs binary classification on fₗ for each ICD code l.

Label-wise feature extraction: Given an input clinical document x containing n words, the present system represents it with a matrix X=[w1, w2, . . . , wn] where wi∈R^{d} is the word embedding vector for the i-th word. Each ICD code l has a textual description. To represent l, the present system constructs an embedding vector vₗ by averaging the embeddings of words in the description. The word embedding is shared between input and label descriptions for sharing learned knowledge. Adjacent word embeddings are combined using a one-dimension convolutional neural network (CNN) to get the n-gram text features H=conv(X)∈R^{N×dc}. Then the label-wise attention feature al∈R^{d} for label l is computed by:

sₗ=softmax(tan h(H·Wₐ^{T}+bₐ)·vₗ),aₗ=sₜ^{T}·H for l=1,2, . . . L

where sₗ is the attention scores for all rows in H and al is the attended output of H for label l. Intuitively, al extracts the most relevant information in H about the code l by using attention. Each input then has in total L attention feature vectors for each ICD code.

Low-shot latent feature generation with WGAN-GP: For a low-shot code l, the code label yₗ for any training data example is yₗ=0 and the binary classifier gi for code assignment is never trained (trained only a few times) with data examples with yₗ=1 due to the dearth of such data. The present system uses GANs to improve low-shot ICD coding by generating pseudo data examples in the latent feature space of medical documents for low-shot codes and fine-tuning the code-assignment binary classifiers using the generated latent features.

More specifically, the present system uses the Wasserstein GAN with gradient penalty (WGAN-GP) to generate code-specific latent features conditioned on the textual description of each code. To condition on the code description, the present system uses a label encoder function C that maps the code description to a low-dimension vector c. In an embodiment, cₗ=C(l). The generator, G:Z×C→F, takes in a random Gaussian noise vector z∈Z and an encoding vector c∈C of a code description to generate a latent feature f̅=G(z, c) for this code. The discriminator or critic, D:F×C→R, takes in a latent feature vector f (either generated by WGAN-GP or extracted from real data examples) and the encoded label vector c to produce a real-valued score D(f, c) representing how realistic f is. The WGAN-GP loss is:

$$\text{?} = \text{?} [ D ( f , c ) ) ] - \text{?} [ D ( \text{?} , c ) ) ] + λ \cdot {\text{?} [ {( ‖ ∇ D ( \text{?} , c ) ) ‖}_{2} - 1 )}^{2} ]$$

$$\text{?} \text{indicates text missing or illegible when filed}$$

Where f=α·f+(1−α)·f̅ with α˜U(0, 1) and λ is the gradient penalty coefficient. WGAN-GP can be learned by solving the minimax problem: minG maxD LWGAN.

Label encoder: The function C is an ICD-code encoder that maps a code description to an embedding vector. For a code l, the present system first uses an LSTM to encode the sequence of M words in the description into a sequence of hidden states [e1, e2, . . . , eM]. Then the present system performs a dimension-wise max-pooling over the hidden state sequence to get a fixed-sized encoding vector el. Finally, the present system obtains the eventual embedding cₗ=cl=el∥gl of code l by concatenating el with gl which is the embedding of l produced by the graph encoding network. Cl contains both the latent semantics of the description (in el) as well as the ICD hierarchy information (in gl).

Keywords reconstruction loss: To ensure the generated feature vector f captures the semantic meaning of code l, the present system encourages f to be able to well reconstruct the keywords extracted from the clinical notes associated with code l. For each input text x labelled with code l, the present system extracts the label-specific keyword set Kl={w1, w2, . . . , wk} as the set of most similar words in x to l, where the similarity is measured by cosine similarity between word embedding in x and label embedding vl. Let Q be a projection matrix, K be the set of all keywords from all inputs and π(⋅,⋅) denote the cosine similarity function, the loss for reconstructing keywords given the generated feature is as following:

$$\text{?} = - log P ( \text{?} \text{\textbar} \text{?} ) \approx - \text{?} π ( \text{?} , \text{?} ) \cdot log P ( \text{?} \text{\textbar} \text{?} ) = - \text{?} π ( \text{?} , \text{?} ) \cdot log \frac{exp ( \text{?} \cdot \text{?} )}{\text{?} exp ( \text{?} \cdot \text{?} )}$$

$$\text{?} \text{indicates text missing or illegible when filed}$$

Discriminating low-shot codes using ICD hierarchy: In the current WGAN-GP framework, the discriminator cannot be trained on low-shot codes due to the lack of real positive features. In order to include low-shot codes during training, the present system utilizes the ICD hierarchy and use f^{sib}, the latent feature extracted from real data of the nearest sibling l^{sib} of a low-shot code l, for training the discriminator. The nearest sibling code is the closest code to l that has the same immediate parent. This formulation would encourage the generated feature f to be close to the real latent features of the siblings of l and thus f can better preserve the ICD hierarchy. More formally, let c^{sib}=C(l^{sib}) the present system presents the following modification to LWGAN for training low-shot codes:

$$\text{?} = \text{?} [ π ( c , \text{?} ) \cdot D ( \text{?} , c ) ] - \text{?} [ π ( c , \text{?} ) \cdot D ( \text{?} , c ) ] + λ \cdot \text{?} [ {( {‖ ∇ D ( \text{?} , c ) ‖}_{1} - 1 )}^{2} ]$$

$$\text{?} \text{indicates text missing or illegible when filed}$$

The loss term by the cosine similarity π(c, c^{sib}) is to prevent generating the exact nearest sibling feature for the low-shot code l. After adding low-shot codes to training, the full learning objective becomes:

$${min}_{G} {max}_{D} \text{?} + \text{?} + β \cdot \text{?}$$

$$\text{?} \text{indicates text missing or illegible when filed}$$

Multi-label classification: For each code l, the binary prediction y\hat{ }l is generated by:

fₗ=rectifier(Wₒ·aₗ+bₒ),ŷₗ=σ(gₜ^{τ}·fₜ)

The present system utilizes graph gated recurrent neural networks (GRNN) to encode the classifier gl. Let V(l) denote the set of adjacent codes of l from the ICD tree hierarchy and t be the number of times the present system propagates the graph, the classifier gl=gₗ^{t} is computed by:

$$\text{?} = \text{?} , \\ \text{?} = \frac{1}{\vert V ( l ) \vert} \text{?} , \\ \text{?} = CRUCell ( \text{?} , \text{?} )$$

$$\text{?} \text{indicates text missing or illegible when filed}$$

where GRUCell is a gated recurrent unit. The weights of the binary code classifier are tied with the graph encoded label embedding gi so that the learned knowledge can also benefit low-shot codes since label embedding computation is shared across all labels. The loss function for training is multi-label binary cross-entropy:

$$\text{?} ( y , \text{?} ) = - \text{?} [ \text{?} log ( \text{?} ) + ( 1 - \text{?} ) log ( 1 - \text{?} ) ]$$

$$\text{?} \text{indicates text missing or illegible when filed}$$

As mentioned above, the distribution of ICD codes is extremely long-tailed. To counter the label imbalance issue, the present system adopts label-distribution-aware margin (LDAM), where the present system subtracts the logit value before sigmoid function by a label-dependent margin Δl:

yₗ^{a}=σ(gₜ^{τ}·fₜ−1(yₗ=1)Δₗ)

The LDAM loss is thus: L_{LDAM}=L_{BCE}(y,ŷ^{m}).

Fine-tuning on generated features: After WGAN-GP is trained, the present system fine-tunes the pre-trained classifier gₗ from the baseline model with generated features for a given low-shot code l. The present system uses the generator to synthesize a set of f̅ and label them with y=1 and collect a set off from training data with yₗ=0 using the baseline model as a feature extractor. The present system fine-tunes gₗ on this set of labelled feature vectors to get the final binary classifier for a given low-shot code l.

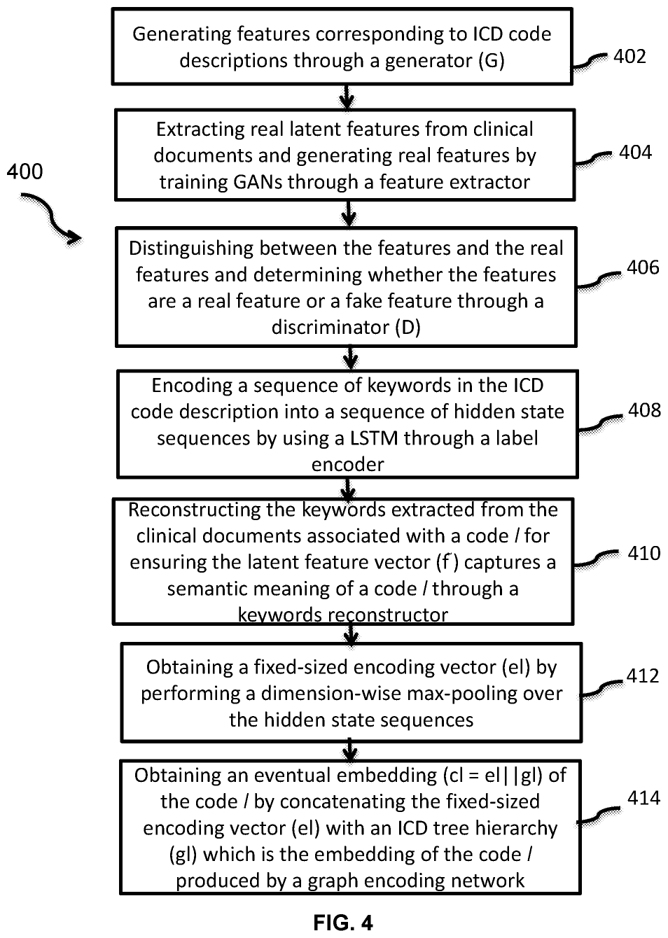

FIG. 4 illustrates a flowchart 400 of the method for classifying a plurality of clinical records into International Classification of Diseases (ICD) codes, in accordance with an alternative embodiment of the present invention. The method includes step 402 of generating one or more features corresponding to one or more ICD code descriptions through a generator (G). The method includes the step 404 of extracting one or more real latent features from a plurality of clinical documents and generating one or more real features by training a plurality of generative adversarial networks (GANs) through a feature extractor. The generator (G) synthesizes the real features after the GANs are trained and calibrates or fine-tunes a binary code classifier with the real latent features generated by the feature extractor for a low-shot ICD code l. In an embodiment, the binary code classifier is encoded by a graph gated recurrent neural networks (GRNN). The GANs improve the low-shot ICD code l by generating a plurality of pseudo data examples in a latent feature space of the clinical documents for the low-shot ICD codes l. The feature extractor generates one or more code-specific latent features conditioned on a textual description of each ICD code description by using a Wasserstein GAN with a gradient penalty (WGAN-GP). The Wasserstein GAN with gradient penalty (WGAN-GP) generates a latent feature vector (f). The method includes the step 406 of distinguishing between the features generated by the generator (G) and the real features generated by the feature extractor and determining whether the features are a real feature or a fake feature through a discriminator (D). The method includes the step 408 of encoding a sequence of a plurality of keywords (M words) in the ICD code description into a sequence of one or more hidden state sequences by using a long short-term memory (LSTM) through a label encoder. The method includes the step 410 of reconstructing the keywords extracted from the clinical documents associated with a code l for ensuring the latent feature vector (f) captures a semantic meaning of a code l through a keywords reconstructor. The method includes step 412 of obtaining a fixed-sized encoding vector (el) by performing a dimension-wise max-pooling over the hidden state sequences through the label encoder.

The method includes the step of obtaining 414 an eventual embedding (cl=of the code l by concatenating the fixed-sized encoding vector (el) with an ICD tree hierarchy (gl) which is the embedding of the code l produced by a graph encoding network through the label encoder. In an embodiment, the eventual embedding (cl) includes a latent semantics of the description (in el) and the ICD tree hierarchy (in gl).

Thus the present system and method provide an efficient, simpler, and more elegant framework that provides an adversarial generative model AGM-HT for automatic ICD coding. The AGM-HT generates latent features conditioned on the code descriptions and fine-tunes the low-shot ICD code assignment classifiers. The present system and method exploit the hierarchical structure of ICD codes to generate semantically meaningful features for low-shot codes without any labelled data. The AGM-HT includes a pseudo cycle generation architecture to guarantee the semantic consistency between the synthetic and real features by reconstructing the relevant keywords in input documents. Further, the present system and method improve the F1 score from nearly 0 to 20.91% for the low-shot codes and AUC score by 3% (absolute improvement) on a MIMIC-III dataset from the previous state of the arts. The AGM-HT improves the performance of few-shot codes with a handful of labelled data.

While embodiments of the present invention have been illustrated and described, it will be clear that the invention is not limited to these embodiments only. Numerous modifications, changes, variations, substitutions, and equivalents will be apparent to those skilled in the art, without departing from the scope of the invention, as described in the claims.

1. A system to classify a plurality of clinical records into International Classification of Diseases (ICD) codes, the system comprising:
one or more processor(s); and
a memory communicatively coupled to the processor(s), wherein the memory stores instructions executed by the processor, wherein the memory comprising:
a generator (G) to generate one or more synthetic features corresponding to one or more ICD code descriptions;
a feature extractor to extract one or more real latent features from a plurality of clinical documents and generates one or more real features by training a plurality of generative adversarial networks (GANs), wherein the generator (G) generates synthesized features after the GANs are trained and calibrate a binary code classifier with the real latent features generated by the feature extractor for a low-shot ICD code l, wherein the GANs improve the low-shot ICD code l by generating a plurality of pseudo data examples in a latent feature space of the clinical documents for the low-shot ICD codes l, wherein the generator (G) generates one or more code-specific latent features conditioned on a textual description of each ICD code descriptions by using a Wasserstein GAN with gradient penalty (WGAN-GP), wherein the Wasserstein GAN with gradient penalty (WGAN-GP) generates a latent feature vector (f̅);
a discriminator (D) to distinguish between the synthesized features generated by the generator (G) and the real features generated by the feature extractor and determines whether the features are the real features generated by the feature extractor or the synthetic features generated by the generator (G);
a label encoder to encode a sequence of a plurality of keywords in the ICD code description into a sequence of one or more hidden state sequences by using a long short-term memory (LSTM); and
a keywords reconstructor to reconstruct the keywords extracted from the clinical documents associated with a code l to ensure the latent feature vector (f̅) captures a semantic meaning of a code l.

2. The system according to claim 1, wherein the label encoder obtains a fixed-sized encoding vector (el) by performing a dimension-wise max-pooling over the hidden state sequences.

3. The system according to claim 1, wherein the label encoder obtains an eventual embedding (cl=el∥gl) of the code l by concatenating the fixed-sized encoding vector (el) with an ICD tree hierarchy (gl) which is the embedding of the code l produced by a graph encoding network.

4. The system according to claim 3, wherein the eventual embedding (cl) comprises a latent semantics of the description (in el) and the ICD tree hierarchy (in gl).

5. The system according to claim 1, wherein the binary code classifier is encoded by a graph gated recurrent neural networks (GRNN).

6. A method for classifying a plurality of clinical records into International Classification of Diseases (ICD) codes, the method comprising steps of:
generating, by one or more processors, one or more synthetic features corresponding to one or more ICD code descriptions through a generator (G);
extracting, by the processors, one or more real latent features from a plurality of clinical documents and generating one or more real features by training a plurality of generative adversarial networks (GANs) through a feature extractor, wherein the generator (G) generates synthesized features after the GANs are trained and calibrates a binary code classifier with the real latent features generated by the feature extractor for a low-shot ICD code l, wherein the GANs improve the low-shot ICD code l by generating a plurality of pseudo data examples in a latent feature space of the clinical documents for the low-shot ICD codes 1, wherein the generator (G) generates one or more code-specific latent features conditioned on a textual description of each ICD code descriptions by using a Wasserstein GAN with gradient penalty (WGAN-GP), wherein the Wasserstein GAN with gradient penalty (WGAN-GP) generates a latent feature vector (f);
distinguishing, by the processors, between the synthesized features generated by the generator (G) and the real features generated by the feature extractor and determining whether the features are the real features generated by the feature extractor or the synthetic features generated by the generator (G) through a discriminator (D);
encoding, by the processors, a sequence of a plurality of keywords in the ICD code description into a sequence of one or more hidden state sequences by using a long short-term memory (LSTM) through a label encoder; and
reconstructing, by the processors, the keywords extracted from the clinical documents associated with a code l for ensuring the latent feature vector (f̅) captures a semantic meaning of a code l through a keywords reconstructor.

7. The method according to claim 6 comprising a step of obtaining, by the processors, a fixed-sized encoding vector (el) by performing a dimension-wise max-pooling over the hidden state sequences through the label encoder.

8. The method according to claim 6 comprising a step of obtaining, by the processors, an eventual embedding (cl=el∥gl) of the code l by concatenating the fixed-sized encoding vector (el) with an ICD tree hierarchy (gl) which is the embedding of the code l produced by a graph encoding network through the label encoder.

9. The method according to claim 8, wherein the eventual embedding (cl) comprises a latent semantics of the description (in el) and the ICD tree hierarchy (in gl).

10. The method according to claim 6, wherein the binary code classifier is encoded by a graph gated recurrent neural networks (GRNN).
